(19) United States
(12) Patent Application Publication
(10) Pub. No.: US 2008/0127974 A1
(43) Pub. Date: Jun. 5, 2008

(54) DISPENSING MEDICAMENT FROM A CAVITY

(75) Inventor: Orest Lastow, Lund (SE)

Correspondence Address: FISH & RICHARDSON P.C., P.O BOX 1022, MINNEAPOLIS, MN, 55440-1022, US

(73) Assignee: AstraZeneca AB

(21) Appl. No.: 11/934,557

(22) Filed: Nov. 2, 2007

Related U.S. Application Data
(63) Continuation-in-part of application No. PCT/SE2006/000520, filed on Apr. 28, 2006.

(30) Foreign Application Priority Data
May 2, 2005 (SE) 0501010-3

(51) Int. Cl. A61M 16/00; B65D 83/00; B32B 37/00; B65D 83/04

(52) U.S. Cl. 128/203.21; 206/531; 221/1; 222/1; 156/69

(57) ABSTRACT

An arrangement for opening a cavity sealed by a sealing sheet the cavity housing a medicament, the arrangement comprising a separating element bondable to the outer surface of the sealing sheet above said cavity, and guiding means for guiding said separating element away from said cavity, whereby the sealing sheet lid covering said cavity and bonded to said separating element is completely separated from the rest of the sealing sheet and removed, and the cavity is opened. A pack for doses of medicament, comprising at least one cavity for housing a dose of medicament, the cavity being sealed by a sealing sheet, where the outer surface of the sealing sheet above the cavity is bonded to the above-mentioned separating element. A dispensing device for dispensing doses of medicament, comprising a dispenser outlet, the dispensing device comprising said arrangement for opening a cavity sealed by a sealing sheet.

### CROSS-REFERENCE TO RELATED APPLICATION

This application is a continuation-in-part of PCT International Application No. PCT/SE2006/000520, filed Apr. 28, 2006, incorporated herein by reference, which claims the priority of Swedish Patent Application No. 0501010-3, filed on May 2, 2005.

### TECHNICAL FIELD

This invention relates to methods and apparatus for dispensing medicament contained in one or more sealed cavities.

### BACKGROUND

Today supply and distribution of medicament is accomplished in many different ways. Within health care more and more is focused on the possibility to dose and distribute medicaments in the form of powder directly to the lungs of a user by means of a dispensing device, for example an inhalation device, to obtain an efficient and user friendly administration of the specific medicament. In most cases, some form of dosing process is used for preparing the dose to be inhaled. Usually, the doses of medicament are provided in packs having several cavities for housing a dose of medicament. The cavities filled with a dose is subsequently sealed by a sealing shee,t for example a foil of aluminium. These packs are loaded into a dispensing device, in which the foil above the cavity is penetrated and the dose of medicament is released for inhalation by the user. By this sealing, the medicament is well protected before inhalation. There are also other cases where it is suitable to provide doses of medicament in packs having cavities for housing a dose of medicament, which cavities are sealed by a foil.

### SUMMARY

One aspect of the present invention features an arrangement for opening a cavity sealed by a sealing sheet, such as a foil, in which cavity a medicament is housed, which can be used for opening different kinds of cavities or blisters covered by a foil, which opens the cavity or blister without impairing the medicament trough pressure or contamination, completely removes the loose foil parts of the ruptured foil, completely opens the cavity of blister for enabling a completely free release of the medicament, presents a low failure risk, and is easy and inexpensive to produce. Further, another aspect provides a pack for doses of medicament, and a dispensing device for dispensing doses of medicament making use of the arrangement above.

Implementing these aspects can be achieved by providing a medicine dose dispenser including a pack with sealed cavities housing a medicament. A separating element can be bonded to the sealing sheet above the cavity opening. The dispenser may also include an actuating device arranged to posh the separating element towards the cavity and then to pull the separating element away from the cavity. The sealing sheet covering the cavity can be separated from the remainder of the sealing sheet when the separating element is pushed towards the cavity. Then, the separating element and the severed portion of the sealing sheet can be pulled away from the cavity allowing the exposed medicament to be dispensed. In some embodiments, the base of the separating element may have overall lateral dimensions sized to cause the sealing sheet to be severed at the edges of the opening when the separating element is pushed toward the cavity. In some embodiments, the actuating devices is arranged to push the separating element into the cavity. In some embodiments, the actuating device and the separating element may include a latch member or assembly or complimentary locking members configured to engage the other to pull the separating element away from the cavity.

In some embodiments, the medicine dose dispenser may be in the form of an inhaler and include a mouth piece and an actuating device configured to pull the separating element away fan the medicament cavity to expose the medicament for inhalation through the mouth piece.

Another aspect of the invention features a medicine pack including a dose of medicament housed in a sealed cavity. A separating element may be bonded to the sealing sheet above the cavity opening and may have overall lateral dimensions sized to cause the sealing sheet to be severed at the edges of the opening when the separating element is forced toward the cavity. In some embodiments, the separating element may be arranged to be pushed into the cavity. Advantageously, the separating element may include a base facing the sealing sheet and having dimensions complementary to the dimensions of the cavity opening. In addition, the separating element may include a latching device for engaging an actuating device configured to pull the separating element away from the cavity opening. In one embodiment, the separating element may include opposing fingers extending away from the cavity and configured to latch onto a cam member prior to being pulled away from the cavity opening.

Another aspect of the invention features a method of dispensing a dose of medicament by grasping a dispenser including a separating element bonded to a sealing sheet above a cavity opening, forcing the separating element into the cavity, pulling the separating element and a severed portion of the sealing sheet away from the cavity to expose the medicament, and then dispensing the medicament from the cavity. The use of the separating element may facilitate opening of a package, for example when used by a user with weak hands, like an old or a rheumatic person. In some embodiments, the separating element may be bonded to the sealing sheet above the cavity opening before forcing the separating element into the cavity, e.g., by the user. In some embodiments, the separating element may be bonded to the sealing sheet by a manufacturer of the dispenser. In some embodiments, the method includes inhaling the dispensed medicament through a mouth piece of the dispenser.

Another aspect of the invention features a method of producing a medicine pack by bonding a sealing sheet across the opening of a cavity housing a dose of medicine and attaching a separating element to the sealing sheet above the opening. The separating element may be configured to sever the sealing sheet at the edges of the opening when pushed toward the cavity.

Some embodiments include packs containing multiple discrete doses of medicine in sealed cavities and multiple separating elements bonded to the sealing sheet above each cavity opening.

Various embodiments described herein provide an effective means of opening of a cavity or blister, without impairing the medicament through pressure or contamination, during which the loose parts of the ruptured sealing sheet are completely removed, whereby no pocket geometries are created which can retain medicament. The arrangement can be used for opening different kinds of cavities or blisters having various designs, even asymmetrical or irregular cavities. Further, the arrangement may present a low failure risk, and can be easy and inexpensive to produce. The cavity can house a powdered, a solid or a liquid medicament. The cavity can be injection moulded or formed as a blister. The separating element can be bonded to the outer surface of the sealing sheet by gluing, induction welding, traditional contact heat welding, ultrasonic welding, or by any suitable method providing a sufficient tensile strength. Advantageously, the sealing sheet can be a foil made of aluminium or plastic, or any other suitable material.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a schematic side view of a first embodiment.

FIG. 2 is a schematic side view of a second embodiment.

FIG. 3 is a schematic side view of the embodiment of FIG. 1 applied to a cavity in the form a blister containing a dose of medicament.

FIG. 4a-4d are schematic side views of a first embodiment of FIG. 1 showing an opening process of a cavity.

FIG. 5 is a schematic perspective view of an embodiment of the pack for doses of medicament.

FIG. 6 is a schematic side view of several embodiments of the separating element, injection moulded as one component.

FIG. 7 is a perspective view of an embodiment of the inhaler.

FIG. 8 is a cross-sectional view of the inhaler of FIG. 7 showing the embodiment of FIG. 1.

### DETAILED DESCRIPTION

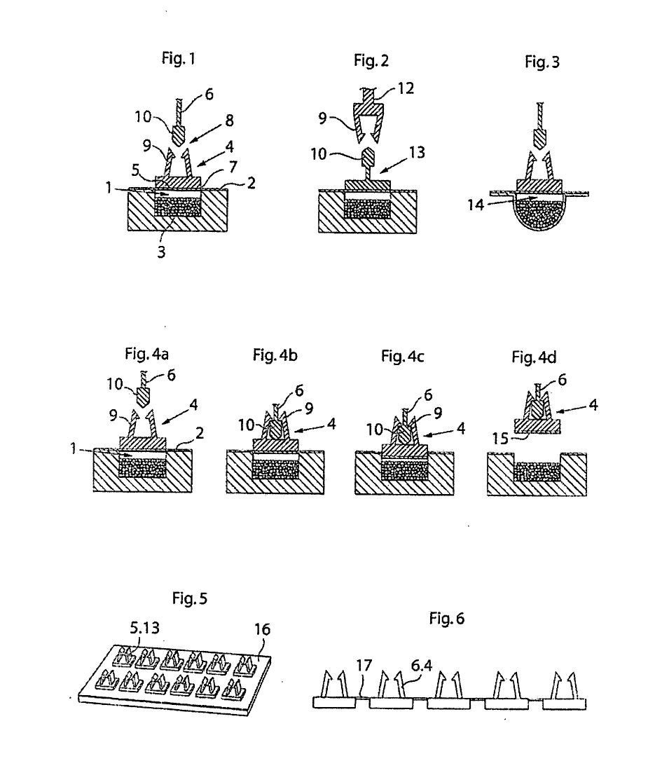

FIG. 1 shows a first embodiment of the arrangement for opening a cavity 1 sealed by a sealing sheet 2 in the form of an aluminium foil, the cavity 1 housing a medicament 3. The arrangement comprises a separating element 4 in the form of a punch head having a flat base. The flat base of the separating element 4 is bonded to the outer surface 5 of the foil above said cavity 1 by adhesion means. Further, the arrangement comprises guiding means 6 for guiding said separating element 4. The guiding means 6 is arranged to push the separating element 4 towards the cavity 1, to depress the separating element 4 into said cavity 1, during said pushing action, and to guide the separating element 4 away from said cavity 1, whereby the entire foil lid covering said cavity 1 and bonded to said separating element 4 is completely separated from the rest of the foil 2 and removed, and the cavity 4 is opened. The base of the separating element 4 facing the foil is provided with sharp edges 7, whereby the foil 2 is easily cut when the separating element 4 is pushed towards the cavity 1. Further, the base of the separating element 4 facing the cavity 1 is complementary to the opening of the cavity 1. The guiding means 6 is in the form of a shaft initially positioned above the separating element. The guiding means 6 and the separating element 4 comprise locking means 8 for locking the guiding means 6 into engagement with the separating element 4 in the opening operation. The locking means $ comprise a resilient claw member 9 provided on the separating element 4, and a tapered tip member 10 with shoulders provided on the guiding means 6. The claw portions of the claw member 9 are arranged to engage the shoulders of the tip member 10, whereby the separating element 4 is locked to the guiding means 6.

FIG. 2 shows a second embodiment of the arrangement where the locking means are inverted, the claw member 9 being provided on the guiding means 12 and the tapered tip member 10 being provided on the separating element 13.

FIG. 3 shows the arrangement of FIG. 1 applied to a cavity in the form a blister 14 containing a dose of medicament.

FIG. 4a-4d show the opening process of a cavity 1 by means of the arrangement of FIG. 1. In FIG. 4a, the guiding means 6 is positioned above the separating element 4. In FIG. 4b, the guiding means 6 has been lowered into the separating element 4, whereby the claw portions of the claw member 9 engages the shoulders of the tip member 10, and the guiding means 6 is locked into engagement with the separating element 4. The guiding means 6 pushes the separating element 4 towards the cavity 1, and in FIG. 4c, the guiding means 6 has depressed the separating element 4 into said cavity 1, whereby the entire foil lid covering said cavity is separated from the rest of the foil 2. In FIG. 4d, the guiding means 6 has removed the separating element 4 away from the cavity, and consequently also the entire foil lid 15 which previously covered the cavity 1, whereby the cavity 1 is completely opened.

FIG. 5 shows an embodiment of the pack for doses of medicament comprising two longitudinal rows of cavities for housing a dose of medicament, each cavity being sealed by a foil 16. The outer surface of the foil 16 above each cavity is bonded to a separating element 5.13, the separating element 5.13 being arranged to be guided away from a certain cavity, whereby the foil lid covering said cavity and bonded to said separating element 5.13 is completely separated from the rest of the foil 16 and removed, and the cavity is opened.

FIG. 6 shows several embodiments of the separating element, injection moulded as one component. The separating elements 6.4 are connected to each other by mould ribs 17, which can be cut away in the same process as the bonding.

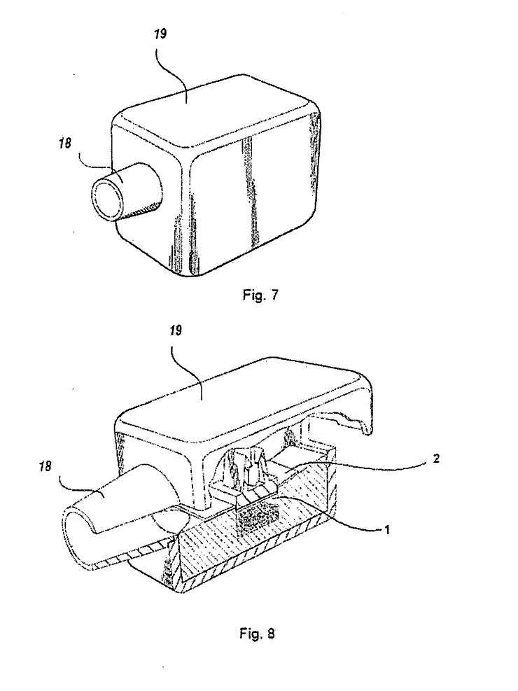

FIG. 7 shows a perspective view of an embodiment of the inhaler. The mouth piece 18 is connected to inhaler housing 19.

FIG. 8 is a cross-sectional view of the inhaler of FIG. 7. In FIG. 8, the arrangement of FIG. 1 is shown housed inside the inhaler housing 19. The separating element is shown depressed into the cavity 1, whereby the entire foil lid covering the cavity is separated from the rest of the foil 2,

A number of embodiments of the invention have been discribed. Nevertheless, it will be understood that various modifications may be made without departing from the spirit and scope of the invention. For example, the separating element and actuating device may be injection moulded as a single piece. Accordingly, other embodiments are within the scope of the following claims.

What is claimed is:

1. A medicine dose dispenser comprising:
a pack, the pack comprising:
a support member defining a cavity having an opening at a top surface of the support member, the cavity housing a dose of medicament therein;
a sealing sheet for covering the cavity; and
a separating element bonded to the sealing sheet and disposed above the cavity; and
an actuating device arranged to push the separating element towards the cavity, whereby the sealing sheet covering the cavity is separated from a remainder of the sealing sheet, and men to pull the separating element away from the cavity.

2. The medicine dose dispenser of claim 1, wherein the actuating device pushes the separating element into the cavity.

3. The medicine dose dispenser of claim 1, wherein the separating element comprises a base facing the sealing sheet, the base having overall lateral dimensions sized to cause the sealing sheet to be severed at edges of the opening when the separating element is pushed toward the cavity.

4. The medicine dose dispenser of claim 1, wherein the separating element comprises a first locking member, and wherein the actuating device comprises a second locking member complimentary to the first locking member and configured to engage the first locking member to pull the separating element away from the cavity.

5. The medicine dose dispenser of claim 1 in the form of an inhaler.

6. The medicine dose dispenser of claim 1, wherein the actuating device comprises a latching member that engages the separating element to pull the separating element away from the cavity.

7. The medicine dose dispenser of claim 1, wherein the separating element and the actuating device comprise respective cooperating portions of a latch assembly that engages to pull the separating element away from the cavity.

8. The medicine dose dispenser of claim 1, wherein the cavity is one of a plurality of cavities defined by the support member and sealed by the sealing sheet, and the separating element is one of a plurality of separating elements, each cavity having a respective separating element bonded to the sealing sheet and disposed above the respective cavity opening.

9. A medicine pack comprising:
a support member defining a cavity having an opening at a top surface of the support member, the cavity housing a dose of medicament therein;
a sealing sheet bonded to the top surface and extending across the opening of the cavity; and
a separating element bonded to the sealing sheet and disposed above the opening, the separating element having overall lateral dimensions sized to cause the sealing sheet to be severed at edges of the opening when the separating element is forced toward the cavity.

10. The medicine pack of claim 9, wherein the separating element is arranged to be pushed into the cavity.

11. The medicine pack of claim 9, wherein the separating element comprises a base facing the sealing sheet, the dimensions of the base complementary to the dimensions of the cavity opening.

12. The medicine pack of claim 9, wherein the separating element comprises a latching device for engaging an actuating device, the actuating device configured to pull the separating element away from the cavity opening.

13. The medicine pack of claim 9, wherein the separating element comprises opposing fingers extending away from the cavity and configured to latch on to a cam member.

14. An inhaler comprising:
a mouth piece;
a medicine pack comprising:
a support member defining a cavity having an opening at a top surface of the support member, the cavity housing a dose of medicament therein;
a sealing sheet bonded to the top surface and extending across the opening of the cavity; and
a separating element bonded to the sealing sheet and disposed above the opening, the separating element having overall lateral dimensions sized to cause the sealing sheet to be severed at edges of the opening when the separating element is forced toward the cavity; and
an actuating device arranged to push the separating element towards the cavity, whereby the sealing sheet covering the cavity is separated from a remainder of the sealing sheet, and then to pull the separating element away from the cavity to expose the medicament for inhalation through the mouth piece.

15. A method of dispensing a dose of medicament, the method comprising:
grasping a dispenser including a separating element bonded to a sealing sheet bonded to a top surface of a support member defining a cavity having an opening at the top surface, the cavity housing a dose of medicament therein, the sealing sheet extending across the opening and the separating element disposed above the opening;
forcing the separating element into the cavity, whereby the sealing sheet is severed from a remainder of the sealing sheet at edges of the cavity opening;
pulling the separating element and a severed portion of the sealing sheet away from the cavity to expose the medicament; and then
dispensing the medicament from the cavity.

16. The method of claim 15 further comprising, prior to forcing the separating element into the cavity, bonding the separating element to the sealing sheet above the cavity opening.

17. The method of claim 15 further comprising inhaling the dispensed medicament through a mouth piece of the dispenser.

18. A method of producing a medicine pack, the method comprising:
bonding a sealing sheet across a top surface of a support member defining a cavity having an opening at the top surface, the cavity housing a dose of medicament therein; and
attaching a separating element to the sealing sheet above the opening, the separating element configured to sever the sealing sheet at edges of the opening when pushed toward the cavity.
